(19) United States
(12) Patent Application Publication
(10) Pub. No.: US 2017/0159189 A1
(43) Pub. Date: Jun. 8, 2017

(54) METHOD FOR PRODUCING 1,5-DIAMINONAPHTHALENE AND 1-AMINO-5-NITRONAPHTHALENE BY DIRECT ELECTROCHEMICAL AMINATION OF NON-ACTIVATED OR DEACTIVATED AROMATIC SYSTEMS ON BORON-DOPED DIAMOND ANODES

(71) Applicant: Covestro Deutschland AG, Leverkusen (DE)

(72) Inventors: Frank Richter, Leverkusen (DE); Heike Heckroth, Odenthal (DE); Reinhard Halpaap, Odenthal (DE); Siegfried R. Waldvogel, Gau-Algesheim (DE); Sebastian Herold, Offenbach am Main (DE); Hartmut Nefzger, Pulheim (DE)

(73) Assignee: Covestro Deutschland AG, Leverkusen (DE)

(21) Appl. No.: 15/309,875

(22) PCT Filed: May 12, 2015
(86) PCT No.: PCT/EP2015/060438

§ 371 Date: Nov. 9, 2016

(30) Foreign Application Priority Data
May 15, 2014 (EP) 14168447.2

(51) Int. Cl. C25B 3/02; C25B 11/12; C07C 211/58; C07C 209/62; C07D 213/16; C07C 211/59

(52) U.S. Cl. CPC C25B 3/02; C07D 213/16; C07C 211/59; C07C 211/58; C07C 209/62; C25B 11/12

(57) ABSTRACT

The invention relates to a method for oxidative electrochemical amination of non-activated or deactivated aromatic systems by means of boron-doped diamond anodes, and in particular to the production of 1,5-diaminonaphthalene and 1-amino-5-nitronaphthalene according to said method.

### CROSS-REFERENCE TO RELATED APPLICATION

This application is a national stage application (under 35 U.S.C. §371) of PCT/EP2015/060438, filed May 12, 2015, which claims benefit of European Application No. 14168447.2, filed May 15, 2014, both of which are incorporated herein by reference in their entirety.

### FIELD OF THE INVENTION

The targeted and selective preparation of defined isomers is frequently difficult in the case of the second substitution on an aromatic. Isomer mixtures and species having more than two substituents are generally obtained. Thus, the targeted synthesis of naphthalene derivatives which are disubstituted in the 1,5 positions has hitherto not been possible.

### BACKGROUND OF THE INVENTION

1,5-Diaminonaphthalene (2), in particular, is an important intermediate for preparing naphthalene 1,5-diisocyanate (1) (FIG. 1). This aromatic diisocyanate is an important basic building block for high-strength polyurethanes.

The industrial synthesis of naphthalene 1,5-diisocyanate proceeds from naphthalene (3) which is converted into 1,5-naphthalenedisulfonic acid (4). 1,5-Naphthalenedisulfonic acid is converted in an alkali melt into 1,5-dihydroxynaphthalene (5) which is then converted in a Bucherer reaction into the diamine 2. Phosgenation is subsequently carried out to give naphthalene 1,5-diisocyanate (1). This classical synthesis of naphthalene 1,5-diisocyanate is shown in scheme 1.

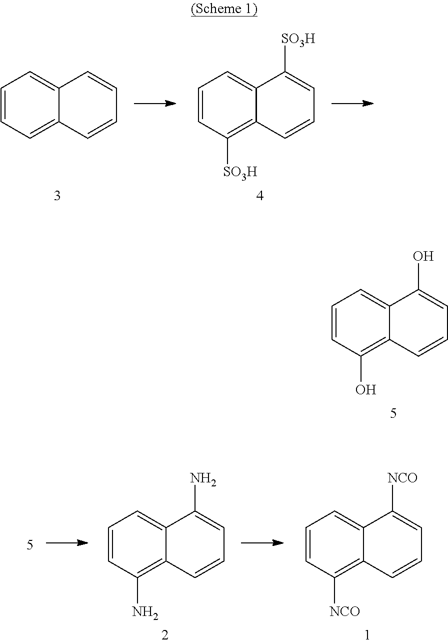

Serious disadvantages of the process described here are the formation of isomer mixtures in the sulfonation of naphthalene. Apart from the desired 1,5 compound, further disulfonic acids and trisulfonic acids are obtained. This is associated with a high loss of yield based on the naphthalene used. The drastic and corrosive conditions in the reaction to form the dihydroxy compound represent further deficiencies of the classical process. The many synthesis steps are associated with time-consuming work-ups of the intermediates. In addition, the many process steps incur high energy costs. Ever scarcer raw materials and the increasing relevance of environmental protection therefore require new synthesis strategies for preparing 1,5-diaminonaphthalene (2).

### SUMMARY OF THE INVENTION

It is an object of the invention to provide environmentally friendly and at the same time inexpensive access to naphthalene derivatives which are disubstituted in the 1,5 positions, in particular to 1,5-diaminonaphthalene. A competitive synthetic preparation of 1,5-diaminonaphthalene, an important precursor for high-strength polyurethanes, can lead to wider use of these robust and long-lived polymers.

These and other advantages and benefits of the present invention will be apparent from the Detailed Description of the Invention herein below.

### BRIEF DESCRIPTION OF THE FIGURES

The present invention will now be described for purposes of illustration and not limitation in conjunction with the figures, wherein:

FIG. 1A provides the structure of naphthalene 1,5-diisocyanate (1)

FIG. 1B shows the structure of 1,5-diaminonaphthalene (2);

FIG. 2A depicts divided Teflon cells in the screening block;

FIG. 2B shows divided H cell with BDD electrodes and magnetic stirrer bars; and

FIG. 3 shows the crystal structure of 1-amino-5-nitronaphthalene (8).

### DETAILED DESCRIPTION OF THE INVENTION

The invention provides a process for the oxidative electrochemical amination of non-activated or deactivated aromatic systems using boron-doped diamond anodes.

For the purposes of the present invention, deactivated aromatic systems are substituted aromatics whose electron density in the ring has been reduced by inductive and/or mesomeric effects of the substituents. Non-activated aromatic systems are unsubstituted aromatics. If a substituted aromatic has both substituents which reduce the electron density in the ring by inductive and/or mesomeric effects and substituents which increase the electron density in the ring by inductive and/or mesomeric effects, such an aromatic comes under the definition according to the invention of a deactivated aromatic system if the electron density in the ring has been reduced overall.

In the process of the invention, naphthalene, naphthalene derivatives substituted in the 1 position and also polycyclic, fused aromatics (>3 rings) and fused heteroaromatics having two or more rings and at least one nitrogen or oxygen atom in the ring are used as deactivated or electron-poor aromatic systems. Naphthalene derivatives substituted in the 1 position include, for example, 1-nitronaphthalene, 1-cyanonaphthalene and 1-alkoxycarbonylnaphthalene or aryloxycarbonyl-naphthalene. Examples of fused aromatics are anthracene, phenanthrene, chrysene, pyrene, perylene and coronene. Examples of heteroaromatics are quinoline, isoquinoline, phthalazine, quinazoline, naphthyridine, acridine, phenazine, phenanthridine and phenanthrolines, e.g. 1,7- or 1,10-phenanthroline.

Preference is given to naphthalene and naphthalene derivatives substituted in the 1 position, with particular preference being given to naphthalene and 1-nitronaphthalene.

The invention provides a process for the oxidative electrochemical amination of
a) 1-nitronaphthalene, 1-cyanonaphthalene, 1-alkoxycarbonylnaphthalene or 1-aryloxy-carbonylnaphthalene in the 5 position, or
b) naphthalene in the 1 and 5 positions
   at boron-doped diamond anodes.

Preference is given to the amination of 1-nitronaphthalene in the 5 position or of naphthalene in the 1 and 5 positions.

Possible amination reagents are first and foremost pyridine and its substituted and fused derivatives such as picolines (2-, 3- and 4-picoline), lutidines (2,3-, 2,4-, 2,5-, 2,6-, 3,4- and 3,5-lutidine) and collidines (2,3,4-, 2,3,5-, 2,3,6-, 2,4,5-, 2,4,6- and 3,4,5-collidine), pyridines having mixed alkyl substituents, for example 5-ethyllutidine and isomers thereof, and also quinoline and isoquinoline.

The invention provides a process for the oxidative electrochemical amination of non-activated or deactivated aromatic systems using boron-doped diamond anodes using pyridine, one or more picoline isomers, one or more lutidine isomers, one or more collidine isomers, quinoline, isoquinoline or mixtures of these compounds as amination reagents.

The invention likewise provides a process for the oxidative electrochemical amination of non-activated or deactivated aromatic systems using boron-doped diamond anodes using pyridine, one or more picoline isomers, one or more lutidine isomers, one or more collidine isomers, one or more pyridine isomers having mixed alkyl substituents, quinoline, isoquinoline or mixtures of these compounds, preferably 5-ethyllutidine and/or one or more isomers thereof, as amination reagents.

The invention provides a process for the electrochemical amination of
c) 1-nitronaphthalene, 1-cyanonaphthalene, 1-alkoxycarbonylnaphthalene or 1-aryloxy-carbonylnaphthalene in the 5 position, or
d) naphthalene in the 1 and 5 positions
   at boron-doped diamond anodes using pyridine, one or more picoline isomers, one or more lutidine isomers, one or more collidine isomers, quinoline, isoquinoline or mixtures of these compounds as amination reagents.

The preferred amination reagent is pyridine.

A number of proton-bearing nucleophiles are in principle possible for the subsequent reaction to set the primary amino group(s) free; mention may be made of water, hydroxide, hydroperoxide, ammonia, amide, hydrazine, hydrazide, hydroxylamine, primary or secondary amines of the formulae NH₂R¹ and NHR¹R², where R¹ and R² are linear or branched, saturated or unsaturated, aliphatic, araliphatic, cycloaliphatic or aromatic radicals which have from 1 to 10 carbon atoms and optionally contain heteroatoms selected from the group consisting of oxygen, sulfur and nitrogen, or in the case of NHR¹ R² together form saturated or unsaturated rings which have from 1 to 6 carbon atoms and optionally contain heteroatoms from the group consisting of oxygen, sulfur and nitrogen.

Preference is given to hydroxide, ammonia, hydrazine, hydroxylamine and piperidine, with particular preference being given to piperidine.

The invention also provides a process for the oxidative electrochemical amination of non-activated or deactivated aromatic systems using boron-doped diamond anodes and subsequent setting-free of the amine from the primary amination product.

The invention also provides a process for preparing 1-amino-5-nitronaphthalene, 1-amino-5-cyano-naphthalene, 1-amino-5-alkoxycarbonylnaphthalene or 1-amino-5-aryloxycarbonylnaphthalene by oxidative electrochemical amination of 1-nitronapthalene, 1-cyanonaphthalene, 1-alkoxycarbonyl-naphthalene or 1-aryloxycarbonylnaphthalene in the 5 position at boron-doped diamond anodes and subsequent setting-free of the amine from the primary amination product.

Preference is given to the preparation of 1-amino-5-nitronaphthalene from 1-nitronaphthalene by this process, and also the 1-amino-5-nitronaphthalene itself prepared via this route.

The invention also provides a process for preparing 1,5-diaminonaphthalene by oxidative electrochemical amination of naphthalene in the 1 and 5 position at boron-doped diamond anodes and subsequent setting-free of the amine from the primary amination product.

The invention likewise provides the last two processes described, in which pyridine is used for the electrochemical amination and piperidine is used for the setting-free of the amine from the primary amination product.

The classical multistage synthesis process to form the diamine 2 is drastically shortened by the direct electrochemical amination of 1-nitronaphthalene in the 5 position and the double amination of naphthalene in the 1,5 positions. Furthermore, losses of yield which in the classical process occur in the 1,5-functionalization of naphthalene due to lack of regioselectivity and/or formation of trisubstituted and higher-substituted intermediates can be avoided.

The electrochemical amination of electron-rich aromatic systems using graphite felt as electrode material has been published by Morofuji et al. 2013 (T. Morofuji, A. Shimizu, J.-I. Yoshida, J. Am. Chem. Soc. 2013, 135, 5000-5003). In contrast thereto, the electrochemical amination of electron-poor, i.e. non-activated or even deactivated, aromatic compounds occurs at anodes composed of boron-doped diamond (BDD) in the present invention. Reaction of the electron-poor starting materials at other anode materials (graphite, vitreous carbon, platinum) is not possible.

In addition, the electrochemical amination of anisole in sulfuric acid/acetonitrile using Ti(IV)/Ti(III) as redox mediator and hydroxylamine as nitrogen source is described in the literature (Y. A. Lisitsin, L. V. Grigor'eva, Russ. J. Gen. Chem. 2008, 78, 1009-1010, Y. A. Lisitsyn, L. V. Grigor'eva, Russ. J. Electrochem. 2009, 45, 132-138, Y. A. Lisitsyn, A. V. Sukhov, Russ. J. Electrochem. 2011, 47, 1180-1185, Y. A. Lisitsyn, A. V. Sukhov, Russ. J. Phys. Chem. 2012, 86, 1033-1034, Y. A. Lisitsyn, A. V. Sukhov, Russ. J. Electrochem. 2013, 49, 91-95, Y. A. Lisitsyn, A. V. Sukhov, Russ. J. Gen. Chem. 2013, 83, 1457-1458).

The electrochemical synthesis of nitroanilines from the corresponding aromatic nitro compounds is likewise known in the literature. Here, nucleophilic attack of a suitable nitrogen nucleophile on an electron-poor nitroaromatic takes place in a reaction step preceding the oxidation. Oxidation of the Meisenheimer complex formed as an intermediate finally gives the substituted aromatic nitro compound. 1-Nitronaphthalene is aminated in the 2 position by this method, 1,3-dinitronaphthalene in the 4 position. Only functionalization of ring A is thus possible (H. Cruz, I. Gallardo, G. Guirado, Green Chem. 2011, 13, 2531-2542, I. Gallardo, G. Guirado, J. Marquet, Eur. J. Org. Chem. 2002, 2002, 251-259).

The electrochemical amination of 1-nitronaphthalene 6 according to the present invention proceeds via the intermediate formation of a radical cation at the BDD anode, which reacts with pyridine or one of its abovementioned derivatives as nucleophile (in scheme 2, depicted by way of example as pyridine). As solvent, it is possible to use, for example, acetonitrile. Since the A ring of 1-nitronaphthalene is more strongly deactivated than the B ring, the attack of pyridine preferably occurs in the 5 position (scheme 2). In this position, the substituents are also at a maximum distance from one another. Setting-free of the amine from the primary naphthylpyridinium system 7 finally gives 1-amino-5-nitronaphthalene 8 (scheme 2), which can be catalytically hydrogenated to form the desired diamine 2.

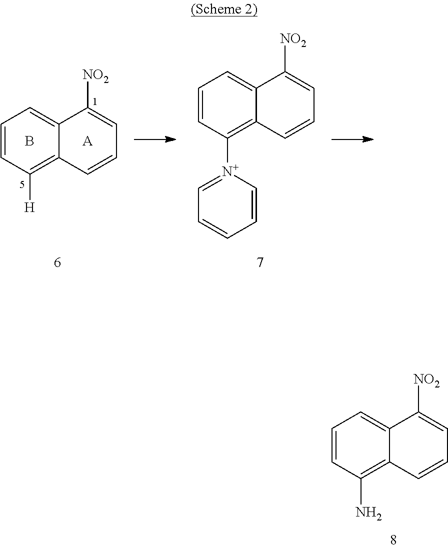

Furthermore, the problem of selective introduction of two amino functions in the 1,5 positions of naphthalene is solved by the double electrochemical amination of naphthalene. The radical cation formed as an intermediate at the BDD anode reacts with pyridine or one of its abovementioned derivatives as nucleophile (in scheme 3, shown by way of example as pyridine). As solvent, it is possible to use, for example, acetonitrile. Since the simple 1-naphthylpyridinium intermediate initially formed has a positive charge in the 1 position, the second pyridine molecule or one of its abovementioned derivatives attacks in the 5 position for electrostatic reasons (in scheme 3, shown by way of example as pyridine) Aminolysis of the 1,5-naphthyldipyridinium compound 7 by means of piperidine gives the desired diamine 2 (scheme 3).

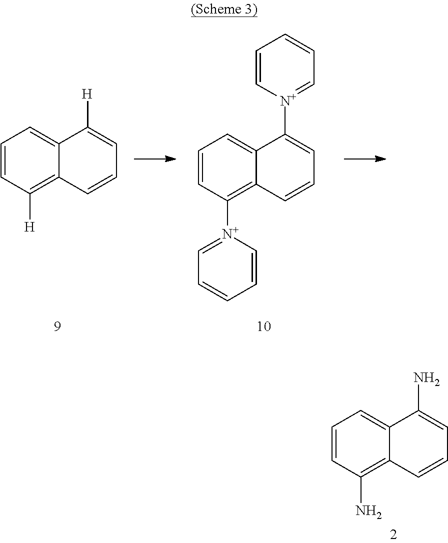 [SMILES: c1ccc2ccccc2c1 ; c1cc[n+](-c2cccc3c(-[n+]4ccccc4)cccc23)cc1 ; Nc1cccc2c(N)cccc12]

This direct method of amination opens up an inexpensive and environmentally friendly alternative to the existing multistage synthesis route.

### EXAMPLES

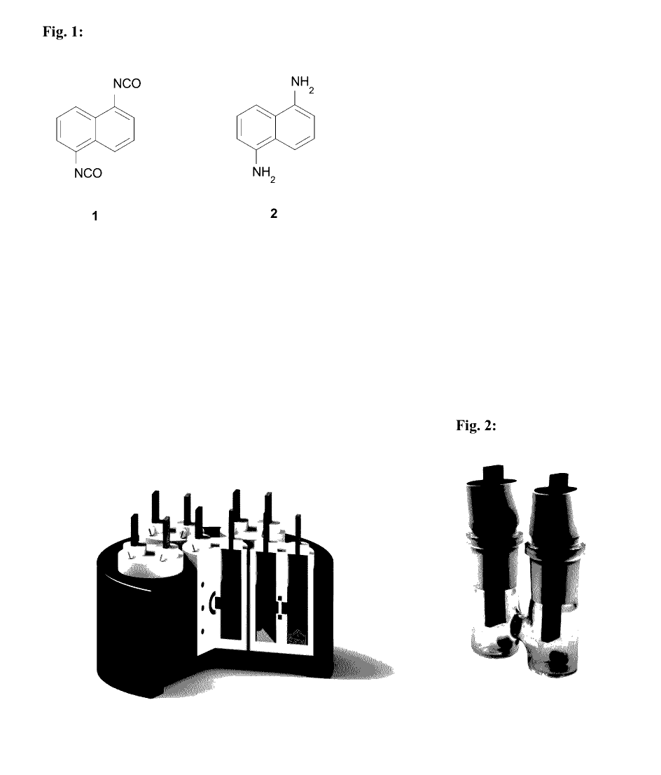

FIGS. 1A and 1B provide the structures of naphthalene 1,5-diisocyanate (1) and 1,5-diaminonaphthalene (2).

FIGS. 2A and 2B show divided Teflon cells in the screening block (2A), divided H cell with BDD electrodes and magnetic stirrer bars (2B). The size of the electrodes: in each case was 10×70 mm; separation of the anode space and the cathode space is in each case was effected by means of a porous separator composed of sintered glass of porosity 4 and having a diameter of 10 mm. The solvent volume in each case was 6 ml. The electrode spacing: was 250 mm (Teflon cell—2A), 350 mm (H cell—2B).

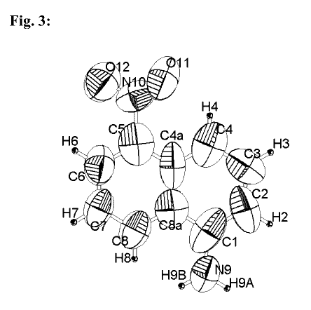

FIG. 3 shows the crystal structure of 1-amino-5-nitronaphthalene (8).

Chromatography:

The preparative liquid-chromatographic separations via “flash chromatography” were carried out at a maximum pressure of 1.6 bar on silica gel 60 M (0.040-0.063 mm) from Macherey-Nagel GmbH & Co, Düren. The separations without pressurization were carried out on silica gel GEDURAN Si 60 (0.063-0.200 mm) from Merck KGaA, Darmstadt. The solvents used as eluents (ethyl acetate (technical grade), cyclohexane (technical grade)) were purified beforehand by distillation on a rotary evaporator.

Thin layer chromatography (TLC) was carried out using ready-to-use PSC silica gel 60 F254 plates from Merck KGaA, Darmstadt. The Rf values (retention factor) are reported as a function of the eluent mixture used. To color the TLC plates, a cerium-molybdatophosphoric acid solution was used as dipping reagent. Cerium-molybdatophosphoric acid reagent: 5.6 g of molybdatophosphoric acid, 2.2 g of cerium(IV) sulfate tetrahydrate and 13.3 g of concentrated sulfuric acid per 200 ml of water.

Gas Chromatography (GC/GCMS):

The gas-chromatographic studies (GC) on product mixtures and pure substances were carried out by means of the gas chromatograph GC-2010 from Shimadzu, Japan. Measurements were carried out on a fused silica capillary column HP-5 from Agilent Technologies, USA (length: 30 m; internal diameter: 0.25 mm; film thickness of the covalently bound stationary phase: 0.25 μm; carrier gas: hydrogen; injector temperature: 250° C.; detector temperature: 310° C.; program: method “hard”: 50° C. initial temperature for 1 min, heating rate: 15° C./min, 290° C. final temperature for 8 min). Gas-chromatographic mass spectra (GCMS) of product mixtures and pure substances were recorded by means of the gas chromatograph GC-2010 combined with the mass detector GCMS-QP2010 from Shimadzu, Japan. Measurements were carried out on a fused silica capillary column HP-1 from Agilent Technologies, USA (length: 30 m; internal diameter: 0.25 mm; film thickness of the covalently bound stationary phase: 0.25 μm; carrier gas: hydrogen; injector temperature: 250° C.; detector temperature: 310° C.; program: method “hard”: 50° C. initial temperature for 1 min, heating rate: 15° C./min, 290° C. final temperature for 8 min; GCMS: temperature of the ion source: 200° C.).

Mass Spectrometry:

All electrospray ionization measurements (ESI+) were carried out on a QTof Ultima 3 from Waters Micromasses, Milford, Mass.

NMR Spectroscopy:

The NMR-spectroscopic studies were carried out on a multinuclear resonance spectrometer model AC 300 or AV II 400 from Bruker, analytical measurement technology, Karlsruhe. d₆-DMSO was used as solvent. The ¹H and ¹³C spectra were calibrated according to the residual content of undeuterated solvent as per the NMR Solvent Data Chart of Cambridge Isotopes Laboratories, USA. The assigning of the ¹H and ¹³C signals was carried out partly with the aid of H,H-COSY (correlation spectroscopy), H,C-HSQC (heteronuclear single quantum coherence spectroscopy) and H,C-HMBC spectra (heteronuclear multiple bond correlation spectroscopy). The chemical shifts are reported as δ values in ppm. The following abbreviations were used for the multiplicities of the NMR signals: s (singlet), bs (broad singlet), d (doublet), t (triplet), q (quartet), m (multiplet), dd (doublet of doublets), dt (doublet of triplets), tq (triplet of quartets). All coupling constants J were reported with the number of included bonds in hertz (Hz). The numbering in the assignment of signals corresponds to the numbering indicated in the formula schemes, which does not have to be in agreement with the IUPAC nomenclature.

Single Crystal Structure Analyses:

The single crystal structure analyses were carried out in the institute for organic chemistry of the Johannes Gutenberg university in Mainz on an instrument model IPDS 2T from STOE & Cie GmbH, Darmstadt.

General Method for Electrochemical Amination:

The electrochemical reaction was carried out in a divided Teflon cell or H cell (FIG. 2). Boron-doped diamond was used as anode material. Platinum was used as cathode material. A solution consisting of the respective aromatic compound (0.1 mol×1⁻¹) and pyridine (2.4 mol×1⁻¹, dry) in a 0.2 molar Bu₄NBF₄/acetonitrile mixture (5 ml, dry) was introduced into the anode space. A solution of trifluoromethanesulfonic acid (0.4 ml) in 0.2 molar Bu₄NBF₄/acetonitrile mixture (5 ml, dry) was introduced into the cathode space. The electrolyses were carried out galvanostatically at 60° C.

After the respective quantities of charge had been reached, the reaction solution was transferred into a 100 ml round-bottom flask and acetonitrile was removed under reduced pressure. 10 ml of acetonitrile were subsequently added together with 1 ml of piperidine and the solution was heated under reflux at 80° C. for 12 hours. The reaction mixture was checked for the amination products by means of GC, DC and GC/MS.

Scheme 2 shows the direct electrochemical amination of 1-nitronaphthalene to 1-amino-5-nitro-naphthalene at boron-doped diamond anodes. The following amounts, materials and process parameters were selected by way of example: 0.5 mmol of 1-nitronaphthalene; 12 mmol of pyridine; 0.2 M Bu₄NBF₄/acetonitrile; solvent: acetonitrile; anode: BDD (1.5 cm²); cathode: platinum (1.5 cm²); temperature: 60° C.; quantity of charge: 144 C; current density: j=10 mA cm⁻². Setting-free of the amine: piperidine; solvent: acetonitrile.

Scheme 3 shows the direct electrochemical amination of naphthalene to 1,5-diaminonaphthalene at boron-doped diamond anodes. The following amounts, materials and process parameters were selected by way of example: 0.5 mmol of 1-nitronaphthalene; 12 mmol of pyridine; 0.2 M Bu₄NBF₄/acetonitrile; solvent: acetonitrile; anode: BDD (1.5 cm²); cathode: platinum (1.5 cm²); temperature: 60° C.; quantity of charge: 288 C; current density: j=10 mA cm⁻². Setting-free of the amine: piperidine; solvent: acetonitrile; temperature: 80° C.; reaction time: 12 hours.

### Example 1 (According to the Invention)

### Preparation of 1-amino-5-nitronaphthalene (8)

According to the above method, 0.09 g (0.53 mmol, 0.04 equivalent) of 1-nitronaphthalene, 0.30 g (0.91 mmol, 0.07 equivalent) of tetrabutylammonium tetrafluoroborate, 1 ml (0.98 g, 12.41 mmol, 1.0 equivalent) of pyridine were dissolved in 5 ml of dry acetonitrile and introduced into the anode space. A solution of 0.3 g (0.91 mmol) of tetrabutylammonium tetrafluoroborate and 0.4 ml of trifluoromethanesulfonic acid in 5 ml of dry acetonitrile was introduced into the cathode space. The electrolysis was carried out in a divided Teflon cell.

Anode: BDD; electrode area: 1.5 cm².

Cathode: platinum; electrode area: 1.5 cm².

Quantity of charge: 144 C.

Current density: j=10 mA cm⁻².

Temperature: 60° C.

After the electrolysis time had elapsed, the solvent was removed under reduced pressure. 10 ml of acetonitrile and 1 ml (0.86 g, 10.00 mmol, 0.8 equivalent) of piperidine were subsequently added. The reaction solution was refluxed at 80° C. for 12 hours. The solvent was removed under reduced pressure and the residue was purified by column chromatography on silica gel using a separation system from Büachi-Labortechnik GmbH. A separation column having a length of 150 mm and a diameter of 40 mm was used and chromatography was carried out at a maximum pressure of 10 bar and a flow rate of 50 ml×min⁻¹. The following solvent gradient was used here: firstly cyclohexane/ethyl acetate 95:5 and the solvent gradient was subsequently increased over the first 40 minutes to cyclohexane/ethyl acetate 3:1 (R_{F}=0.16). 19.6 mg (0.1 mmol, 21%) of a reddish solid were obtained.

¹H-NMR (400 MHz, DMSO): δ (ppm)=6.18 (s, 2 H, NH₂), 6.83 (dd, ³J=7.2 Hz, ⁴J=1.3 Hz, 1 H, H-2), 7.35-7.56 (m, 3 H, H-3,4,7), 8.14 (d, ³J=7.5 Hz, 1 H, H-8), 8.49 (d, ³J=8.5 Hz, 1 H, H-6).
¹³C-NMR (101 MHz, DMSO): δ (ppm)=108.4 (C-4), 108.8 (C-2), 121.8, 123.37 (C-8), 125.4 (C-4a), 128.2 (C-6), 130.6, 146.0 (C-1), 146.6 (C-5).
GC (method hard, HP-5): t_{R}=12.78 min (retention time).
HRMS (High Resolution Mass Spectrometry) calculated for C₁₀H₉N₂O₂: 189.0664; found: 189.0662.

Crystal structure analysis (crystal structure: FIG. 3):

The single crystal was obtained by crystallization of 20 mg of 8 from n-heptane/methanol.

| | |
|---|---|
| Empirical formula | C₁₀H₈N₂O₂ |
| Molecular weight | 188.2 g mol⁻¹ |
| Temperature | −80° C. |
| Radiation | Cu—K_{α} |
| Crystal system, space group | orthorhombic, P na2₁ |
| Lattice constants | a = 12.935(2) Å |
| | b = 16.996(4) Å |
| | c = 3.9407(7) Å |
| Cell volume | V = 866.3(3) Å³ |
| Z, calculated density | 4, 1.443 g cm⁻³ |
| Absorption coefficient | 0.86 mm⁻¹ |
| F(000) | 392 |
| Crystal size | 0.01 × 0.01 × 0.6 mm³ brown needle |
| Θ range of data collection | 2.6-67.8° |
| Reflection range | −15 ≦ h ≦ 12, |
| | −20 ≦ k ≦ 16, −4 ≦ l ≦ 4 |
| Measured/independent reflections | 1501 (Rᵢₙₜ = 0.3445) |
| Discrepancy factor | wR2 = 0.4938 (R1 = 0.1574 for |
| | reflections observed 0.4437 |
| | for all reflections) |
| Quality of fit | S = 0.808 |

The structure data of the compound have been deposited under the designation SHE1332 at the Organischen Institut of the University of Mainz (Dr. D. Schollmeyer, X-ray structure analysis, Duesbergweg 10-14, 55128 Mainz).

### Example 2 (According to the Invention)

Preparation of 1,5-diaminonaphthalene (2)

According to the general method, 0.064 g (0.50 mmol, 0.04 equivalent) of naphthalene, 0.30 g (0.91 mmol, 0.07 equivalent) of tetrabutylammonium tetrafluoroborate, 1 ml (0.98 g, 12.41 mmol, 1.0 equivalent) of pyridine were dissolved in 5 ml of dry acetonitrile and introduced into the anode space. A solution of 0.3 g (0.91 mmol) of tetrabutylammonium tetrafluoroborate and 0.4 ml of trifluoromethanesulfonic acid in 5 ml of dry acetonitrile was introduced into the cathode space. The electrolysis was carried out in a divided H cell.

Anode: BDD; electrode area: 1.5 cm².

Cathode: platinum; electrode area: 1.5 cm².

Quantity of charge: 288 C.

Current density: j=10 mA cm⁻².

Temperature: 60° C.

After the electrolysis time had elapsed, the solvent was removed under reduced pressure. 10 ml of acetonitrile and 1 ml (0.86 g, 10.00 mmol, 0.8 equivalent) of piperidine were subsequently added. The reaction solution was refluxed at 80 C for 12 hours. After the end of the reaction time, the solvent was removed under reduced pressure and the residue was purified by column chromatography on silica gel (cyclohexane/ethyl acetate 2:1, R_{F}=0.39). 0.01 g (0.06 mmol, 10%) of a brown solid was obtained.

¹H-NMR (400 MHz, DMSO): δ (ppm)=5.41 (s, 4 H, NH₂), 6.60 (dd, ³J=7.2 Hz, ⁴J=1.1 Hz, 2 H, H-2), 7.05 (dd, ³J=8.4 Hz, 7.2 Hz, 2 H, H-3) 7.17-7.24 (m, 2 H, H-4).
¹³C-NMR (101 MHz, DMSO): δ (ppm)=107.4 (C-2), 110.0 (C-4), 123.8 (C-4a), 124.3 (C-3), 144.5 (C-1).
GC (method hard, HP-5): t_{R}=11.89 min.
HRMS calculated for C₁₀H₁₁N₂: 159.0922; found: 159.0920.

### MS (EI, 70 eV): m/z (%): 158 (100) [M]^{·+}

This specification has been written with reference to various non-limiting and non-exhaustive embodiments. However, it will be recognized by persons having ordinary skill in the art that various substitutions, modifications, or combinations of any of the disclosed embodiments (or portions thereof) may be made within the scope of this specification. Thus, it is contemplated and understood that this specification supports additional embodiments not expressly set forth herein. Such embodiments may be obtained, for example, by combining, modifying, or reorganizing any of the disclosed steps, components, elements, features, aspects, characteristics, limitations, and the like, of the various non-limiting embodiments described in this specification. In this manner, Applicant(s) reserve the right to amend the claims during prosecution to add features as variously described in this specification, and such amendments comply with the requirements of 35 U.S.C. §112(a), and 35 U.S.C. §132(a).

Various aspects of the subject matter described herein are set out in the following numbered clauses:

1. A process for the oxidative electrochemical amination of non-activated or deactivated aromatic systems using boron-doped diamond anodes.

2. The process as in clause 1, wherein naphthalene, naphthalene derivatives substituted in the 1 position, polycyclic, fused aromatics or fused heteroaromatics having two or more rings and at least one nitrogen or oxygen atom in the ring are used as deactivated or electron-poor aromatic systems.

3. The process as in clause 2, wherein 1-nitronaphthalene, 1-cyanonaphthalene, 1-alkoxycarbonylnaphthalene or 1-aryloxycarbonylnaphthalene is used as naphthalene derivative substituted in the 1 position and anthracene, phenanthrene, chrysene, pyrene, perylene and coronene are used as polycyclic, fused aromatics and quinoline, isoquinoline, phthalazine, quinazoline, naphthyridine, acridine, phenazine, phenanthridine or phenanthrolines are used as heteroaromatics having two or more rings.

4. The process as in clause 1, wherein naphthalene or naphthalene derivatives substituted in the 1 position, preferably naphthalene or 1-nitronaphthalene, are used as deactivated or electron-poor aromatic systems.

5. A process for the oxidative electrochemical amination of
a) 1-nitronaphthalene, 1-cyanonaphthalene, 1-alkoxycarbonylnaphthalene or 1-aryloxycarbonylnaphthalene in the 5 position, or
b) naphthalene in the 1 and 5 positions

at boron-doped diamond anodes.

6. The process as in any of clauses 1 to 5 using pyridine, one or more picoline isomers, one or more lutidine isomers, one or more collidine isomers, quinoline, isoquinoline or mixtures of these compounds as amination reagents.

7. The process as in any of clauses 1 to 5, wherein pyridine is used as amination reagent.

8. The process as in any of clauses 1 to 5 using pyridine, one or more picoline isomers, one or more lutidine isomers, one or more collidine isomers, one or more pyridine isomers having mixed alkyl substituents, quinoline, isoquinoline or mixtures of these compounds, preferably 5-ethyllutidine and/or one or more isomers thereof, as amination reagents.

9. A process for the oxidative electrochemical amination of non-activated or deactivated aromatic systems using boron-doped diamond anodes and subsequent setting-free of the amine from the primary amination product.

10. A process for preparing 1-amino-5-nitronaphthalene, 1-amino-5-cyanonaphthalene, 1-amino-5-alkoxycarbonylnaphthalene or 1-amino-5-aryloxycarbonylnaphthalene by oxidative electrochemical amination of 1-nitronapthalene, 1-cyanonaphthalene, 1-alkoxycarbonylnaphthalene or 1-aryloxycarbonylnaphthalene in the 5 position at boron-doped diamond anodes and subsequent setting-free of the amine from the primary amination product.

11. A process for preparing 1,5-diaminonaphthalene by oxidative electrochemical amination of naphthalene in the 1 and 5 position at boron-doped diamond anodes and subsequent setting-free of the amine from the reaction product.

12. The process as in any of clauses 9 to 11, wherein hydroxide, ammonia, hydrazine, hydroxylamine or piperidine, preferably piperidine, is used for the setting-free of the amine.

13. 1-Amino-5-nitronaphthalene obtainable by a process as in clause 10 or 12.

1. A process comprising conducting oxidative electrochemical amination of one of a non-activated and a deactivated aromatic compound in the presence of one or more boron-doped diamond anodes and one or more amination reagents.

2. The process according to claim 1, wherein the deactivated aromatic compound is selected from the group consisting of naphthalene, naphthalene derivatives substituted in the 1 position, polycyclic, fused aromatics or fused heteroaromatics having two or more rings and at least one nitrogen or oxygen atom in the ring.

3. The process according to claim 2, wherein the naphthalene derivative substituted in the 1 position is selected from the group consisting of 1-nitronaphthalene, 1-cyanonaphthalene, 1-alkoxycarbonylnaphthalene and 1-aryloxycarbonylnaphthalene wherein the polycyclic, fused aromatic is selected from the group consisting of anthracene, phenanthrene, chrysene, pyrene, perylene and coronene and wherein the heteroaromatic having two or more rings is selected from the group consisting of quinoline, isoquinoline, phthalazine, quinazoline, naphthyridine, acridine, phenazine, phenanthridine or phenanthrolines.

4. The process according to claim 1, wherein the deactivated aromatic compound is selected from the group consisting of naphthalene and 1-nitronaphthalene.

5. A process comprising conducting oxidative electrochemical amination of one selected from the group consisting of 1-nitronaphthalene, 1-cyanonaphthalene, 1-alkoxycarbonylnaphthalene, 1-aryloxycarbonyl-naphthalene in the 5 position, or naphthalene in the 1 and 5 positions in the presence of one or more boron-doped diamond anodes.

6. The process according to claim 1, wherein the amination reagent is selected from the group consisting of pyridine, one or more picoline isomers, one or more lutidine isomers, one or more collidine isomers, quinoline, isoquinoline or mixtures of these compounds.

7. The process according to claim 1, wherein the amination reagent is pyridine.

8. The process according to claim 1, wherein the amination reagent is selected from the group consisting of pyridine, one or more picoline isomers, one or more lutidine isomers, one or more collidine isomers, one or more pyridine isomers having mixed alkyl substituents, quinoline, isoquinoline or mixtures of these compounds.

9. A process comprising conducting oxidative electrochemical amination of non-activated or deactivated aromatic compounds in the presence of one or more boron-doped diamond anodes and setting-free of an amine from a primary amination product.

10. A process for preparing one of 1-amino-5-nitronaphthalene, 1-amino-5-cyanonaphthalene, 1-amino-5-alkoxycarbonylnaphthalene or 1-amino-5-aryloxycarbonylnaphthalene comprising conducting oxidative electrochemical amination of one of 1-nitronapthalene, 1-cyanonaphthalene, 1-alkoxycarbonylnaphthalene or 1-aryloxycarbonylnaphthalene in the 5 position in the presence of boron-doped diamond anodes and setting-free an amine from a primary amination product.

11. A process for preparing 1,5-diaminonaphthalene comprising conducting oxidative electrochemical amination of naphthalene in the 1 and 5 position in the presence of boron-doped diamond anodes and setting-free an amine from a reaction product.

12. The process according to claim 9, wherein the step of setting free includes one selected from the group consisting of hydroxide, ammonia, hydrazine, hydroxylamine or piperidine.

13. 1-Amino-5-nitronaphthalene produced by the process according to claim 10.
